(19) United States
(12) Patent Application Publication
(10) Pub. No.: US 2024/0391967 A1
(43) Pub. Date: Nov. 28, 2024

(54) METHODS AND USES FOR NDFIP1 FUSION POLYPEPTIDES IN TREATING NEURODEGENERATIVE DISEASES, BRAIN AND/OR TRAUMATIC AND NON-TRAUMATIC SPINAL CORD INJURIES, AND/OR OPTIC NEUROPATHIES

(71) Applicant: University Health Network, Toronto (CA)

(72) Inventors: Michael George FEHLINGS, Toronto (CA); Mohammad KHAZAEI, Toronto (CA)

(73) Assignee: University Health Network, Toronto, ON (CA)

(21) Appl. No.: 18/255,136

(22) PCT Filed: Dec. 2, 2021
(86) PCT No.: PCT/CA2021/051720

§ 371 Date: May 31, 2023

Related U.S. Application Data
(60) Provisional application No. 63/120,574, filed on Dec. 2, 2020.

(51) Int. Cl. C07K 14/47; A61K 38/00; A61P 25/28; C07K 7/08; C12N 15/63; C12N 15/85

(52) U.S. Cl. CPC C07K 14/4702; A61P 25/28; C07K 7/083; C12N 15/635; C12N 15/85; A61K 38/00; C07K 2319/10; C07K 2319/33; C07K 2319/95

(57) ABSTRACT

Disclosed herein are methods for treating a neurodegenerative disease and/or an optic nerve, brain and/or spinal cord injury using a Ndfip1 fusion polypeptide, a Ndfip1 nucleic acid molecule, a construct or expression cassette comprising the Ndfip1 nucleic acid molecule, and a cell comprising the construct and/or expression the fusion polypeptide.

### CROSS-REFERENCE TO RELATED APPLICATIONS

This application is an International PCT application, which claims priority from U.S. provisional application Ser. No. 63/120,574 filed Dec. 2, 2020, which is incorporated herein by reference in its entirety.

### INCORPORATION OF SEQUENCE LISTING

A computer readable form of the Sequence Listing P63043PC00 (10,164 bytes), submitted via EFS-WEB and created on Dec. 1, 2021, is herein incorporated by reference.

### FIELD

The present disclosure relates to Ndfip1 fusion polypeptides, nucleic acids encoding and cells expressing said fusion polypeptides as well as methods for treating a neurodegenerative disease, brain and/or traumatic and non-traumatic spinal cord injuries, and/or optic nerve injuries, using said Ndfip1 fusion polypeptides and related products.

BACKGROUND

Various cellular and molecular therapies have been tested in animal models of SCI including modulating the PTEN/mTOR pathway which is considered one of the most promising^{1,2,3,4,5,6}. PTEN (phosphatase and tensin homolog), is a negative regulator of the mammalian target of rapamycin (mTOR) pathway and primarily identified as a tumor suppressor. PTEN reduces axon regeneration^{3,2,1,7,8}. Previous studies have demonstrated that treatment with pharmacological inhibitors of PTEN, or knockdown using siRNA, results in a dramatic increase in neurite outgrowth and improves functional recovery after SCI^{9,10}. Conditional deletion of PTEN, in injured CNS neurons promoted robust axon regeneration and enhanced compensatory sprouting of uninjured axons^{1,2,3,1,3.7}. However, PTEN is also crucial for proper function of neurons and neuronal survival. PTEN is required for synapse formation and synaptic plasticity¹¹ and nuclear PTEN is crucial for neuronal survival and neuroprotection, after neuronal damage¹². Complete ablation of PTEN from neurons causes widespread deficits in neuronal growth, synaptogenesis and synaptic plasticity, structure and transmission^{13,14,15,16,17,18}. On the other hand nuclear localization of PTEN is a dynamic process which is associated with neuronal survival^{12,19,20}. Although PTEN is mainly localized to the cytoplasm, in differentiated or resting cells, like neurons, it also resides in the nucleus²¹. This indicates that any intervention for treatment of SCI that completely eliminates PTEN from neurons could be deleterious and presence of a minimum regulated amount of PTEN in neuronal cells is crucial for the proper function of nervous system.

Ubiquitination of proteins is an important regulatory mechanism. It targets proteins for degradation by the proteasome; it also can affect the sorting or trafficking of proteins²². Different studies have shown that the intracellular trafficking of PTEN is regulated by ubiquitination^{23,24}. Ndfip1 is an adaptor protein which regulates PTEN by recruiting the E3 ubiquitin ligase, Nedd4, and enhancing the ubiquitination of PTEN and subsequent nuclear transport of PTEN or its degradation^{19,25,23,26}. Ndfip1 is also required for proper trafficking of PTEN to synaptic terminals. Previous studies have demonstrated that Nedd4-1, through its adaptor Ndfip1, is required for axon development and proper synapse formation²⁷. In cortical brain injury Ndfip1 expression has been shown to be upregulated along with Nedd4, specifically in surviving neurons next to the trauma lesion²⁰.

Previous studies have disclosed methods for promoting differentiation of neural progenitor cells (NPCs) in vitro to oligodendrocytes by treating NPCs with Ndfip1.³⁴

Previous studies have illustrated that inhibition of PTEN by knockdown or knockout stimulates various degrees of axon regrowth.³⁵

There are no known ways to reverse damage to the spinal cord, or optic nerve. Neurodegeneration treatments are also lacking.

Treatments for neurodegenerative diseases and/or optic nerve, brain, and/or spinal cord injuries, are desirable.

### SUMMARY

A first aspect of the invention includes a t Ndfip1 fusion polypeptide comprising a neuron transport moiety, and a Ndfip1 peptide.

In an embodiment, the neuron transport moiety is or comprises a Rabies Virus glycoprotein (RVG)-neuron permeabilization peptide, a translocation domain of diphtheria toxin (DTT), nontoxic C fragment of tetanus toxin (TTC), non-toxic pentameric b chain of the “Cholera toxin” (CTb), Neurotensin (NT) or Tet1 or an analog thereof maintains the ability to facilitate transport into a neuron

In an embodiment, the neuron transport moiety is or comprises a neuron surface receptor ligand.

In an embodiment, the neuron transport moiety is or comprises an antibody, optionally a single domain antibody.

In an embodiment, the neuron transport moiety has the sequence of SEQ ID NO: 1, 2, 3 or 4 or at least 90% sequence identity to any of SEQ ID NO: 1, 2, 3, or 4.

In an embodiment, wherein the antibody targets transferrin receptor (TfR), insulin receptor (IR), p75-NTR, or GT1b.

Another aspect of the disclosure includes a nucleic acid molecule encoding the Ndfip1 fusion polypeptide described herein.

Another aspect of the disclosure includes construct or expression cassette comprising a nucleic acid molecule described herein.

In an embodiment, the construct is a vector comprising the expression cassette.

In an embodiment, the vector is a viral vector, optionally a Herpes Simplex Virus, Adenovirus, Adeno-associated virus (AAV) or retrovirus vector, optionally a lentivirus vector.

In an embodiment, the construct or expression cassette further comprises an inducible promoter.

In an embodiment, the construct or expression cassette further comprises an export signal polynucleotide, optionally encoding any one of SEQ ID Nos: 6 to 23, preferably SEQ ID NO: 7 or 11.

In an embodiment, the inducible promoter is a Tet-On inducible promoter, optionally TRE3G.

Another aspect of the disclosure includes a cell expressing the Ndfip1 fusion polypeptide, optionally wherein the cell comprises a nucleic acid molecule, construct or expression cassette described herein.

In an embodiment, the cell comprises a construct described herein.

In an embodiment, the cell is a neural lineage cell, optionally a neural progenitor cell (NPC). In an embodiment, the NPC is an oligodendrogenic NPC (oNPC). In another embodiment, the NPC is a spinal identity NPC (spNPC). In yet another embodiment, cell is a fibroblast. In another embodiment, the cell is selected from neural stem/progenitor cell, motor-neuron progenitor cell, differentiated neuron, neural stem cell, ventral neural progenitor cell, motor neuron progenitor (MNP), Motor Neural Progenitor Cell (pMN), Neuroepithelial precursor cell, or a central nervous system (CNS) neuronal cell type.

Also provided in another aspect is a therapeutic for use in treating a neurodegenerative disease and/or an optic nerve, brain, and/or spinal cord injury comprising a Ndfip1 fusion polypeptide, nucleic acid molecule, expression cassette or construct or cell described herein.

In an embodiment, the therapeutic comprises a cell described herein.

Another aspect is a method of treating a neurodegenerative disease and/or an optic nerve, brain, and/or spinal cord injury in a subject in need thereof, the method comprising:
a. administering the Ndfip1 fusion polypeptide, nucleic acid molecule, or construct described herein;
b. administering a cell described herein to the subject followed by an inducing agent, wherein the cell comprises an expression cassette or construct with an inducible promoter; or
c. administering an inducing agent to the subject, wherein the subject has previously been administered the cell, wherein the cell administered comprised an expression cassette or construct with an inducible promoter.

In an embodiment, the Ndfip1 fusion polypeptide, nucleic acid molecule, construct or the cell is administered to or proximal to neurons damaged by the neurodegenerative disease and/or the optic nerve, brain, and/or spinal cord injury.

In an embodiment, the method comprises administering a cell described herein to the subject subsequently followed by an inducing agent.

In an embodiment, the inducible promoter is tetracycline responsive promoter, optionally TRE3G and the inducing agent is doxycycline.

In an embodiment, wherein the subject in need thereof has a neurodegenerative disease.

In an embodiment, the neurodegenerative disease is multiple sclerosis (MS), amyotrophic sclerosis (ALS), Alzheimer's disease, Parkinson's Disease, or Huntington's Disease.

In an embodiment, the subject in need thereof has an optic nerve, brain and/or spinal cord injury.

In an embodiment, the subject in need thereof has a spinal cord injury.

In an embodiment, the Ndfip1 fusion polypeptide, nucleic acid molecule, construct or the cell is administered to the subject not earlier than two weeks following the optic nerve, brain and/or spinal cord injury. The Ndfip1 fusion polypeptide, nucleic acid molecule, construct or the cell may be administered to the subject in a composition.

In an embodiment, the subject is a human.

A further aspect is a method of making a cell of described herein, the method optionally comprising the following steps:
a. inserting a nucleic acid molecule or expression cassette described herein into a vector to make a vector construct; and
b. transfecting a cell with the vector construct.

A further aspect is a composition comprising a Ndfip1 fusion polypeptide, nucleic acid molecule or construct or expression cassette, or cell described herein and optionally a pharmaceutically acceptable carrier.

In an embodiment, the nucleic acid molecule, expression cassette or construct is complexed with lipid particle.

In an embodiment the composition comprises a cell described herein and a pharmaceutically acceptable carrier, optionally for use in a method or use described herein.

Also provided in another aspect is use of the Ndfip1 fusion polypeptide, the Ndfip1 nucleic acid molecule, the construct, the composition, or the cell described herein in the manufacture of a medicament for treating a neurodegenerative disease and/or an optic nerve, brain, and/or spinal cord injury.

Also provided in a further aspect is a Ndfip1 fusion polypeptide, Ndfip1 nucleic acid molecule, expression cassette, construct, composition, or cell described herein to treat a neurodegenerative disease and/or an optic nerve, brain, and/or spinal cord injury.

### BRIEF DESCRIPTION OF THE DRAWINGS

An embodiment of the present disclosure will now be described in relation to the drawings in which:

FIGS. 1A and B depict the results of a Western Blot analysis of cytoplasmic, nuclear and synaptosomal fractions from cultured neurons transfected with GFP, Ndfip1 or Nedd4. Antibodies against Actin, lamin and Synaptophysin were used as loading control for each fraction.

FIG. 2 depicts images illustrating overexpression of Ndfip1 in cultured neurons, induced the transport of PTEN to the nucleus.

FIGS. 3A, B, C, and D depict images illustrating that Ndfip1 overexpression in cultured neurons could increase the survival rate of neurons after in vitro injury.

FIG. 4A depicts images illustrating the effect of Ndfip1 and Nedd4 on axon out-growth after injury as compared to a control (GFP). FIG. 4B is a graph illustrating the effect of Ndfip1 and Nedd4 on axon length after injury as compared to a control (GFP).

FIGS. 5A, B, and C depict the effect of Nedd4 on voltage gated sodium channels.

FIG. 6 depicts a graph illustrating axon length depending on the concentration of Ndfip1 secreted into the neurons.

FIGS. 7A, B and C depict constructs for expression of Ndfip1. FIG. 7A depicts a schematic of an expression cassette comprising a Ndfip1 polynucleotide. FIG. 7B depicts a schematic of a vector that comprises the expression cassette of FIG. 7A. FIG. 7C is a schematic of a Ndfip1 fusion polypeptide.

### DETAILED DESCRIPTION

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. For example, the term “a cell” includes a single cell as well as a plurality or population of cells. Generally, nomenclatures utilized in connection with, and techniques of, cell and tissue culture, molecular biology, and protein and oligonucleotide or polynucleotide chemistry and hybridization described herein are those well-known and commonly used in the art (see, e.g., Green and Sambrook, 2012, 4^{th} ed 2014).

As used in this specification and the appended claims, the singular forms “a”, “an” and “the” include plural references unless the content clearly dictates otherwise. Thus, for example, a composition containing “a compound” includes a mixture of two or more compounds. It should also be noted that the term “or” is generally employed in its sense including “and/or” unless the content clearly dictates otherwise.

In understanding the scope of the present disclosure, the term “comprising” and its derivatives, (such as “comprise” and “comprises”), “having” (and any form of having, such as “have” and “has”), “including” (and any form of including, such as “include” and “includes”) or “containing” (and any form of containing, such as “contain” and “contains”), as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The terms “about”, “substantially” and “approximately” as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies.

The definitions and embodiments described in particular sections are intended to be applicable to other embodiments herein described for which they are suitable as would be understood by a person skilled in the art.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

The recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”. For ranges described herein, subranges are also contemplated, for example every, 0.1 increment there between. For example, if the range is 80% to about 90%, also contemplated are 80.1% to about 90%, 80% to about 89.9%, 80.1% to about 89.9% and the like.

The term “cell” as used herein refers to a single cell or a plurality of cells.

The terms “nucleic acid”, “oligonucleotide” as used herein means two or more covalently linked nucleotides. Unless the context clearly indicates otherwise, the term generally includes, but is not limited to, deoxyribonucleic acid (DNA) and ribonucleic acid (RNA), which may be single-stranded (ss) or double stranded (ds). For example, the nucleic acid molecules or polynucleotides of the disclosure can be composed of single-and double-stranded DNA, DNA that is a mixture of single-and double-stranded regions, single-and double-stranded RNA, and RNA that is a mixture of single-and double-stranded regions, hybrid molecules comprising DNA and RNA that may be single-stranded or, more typically double-stranded or a mixture of single- and double-stranded regions. In addition, the nucleic acid molecules can be composed of triple-stranded regions comprising RNA or DNA or both RNA and DNA. The term “oligonucleotide” as used herein generally refers to nucleic acids up to 200 base pairs in length and may be single-stranded or double-stranded. The sequences provided herein may be DNA sequences or RNA sequences, however it is to be understood that the provided sequences encompass both DNA and RNA, as well as the complementary RNA and DNA sequences, unless the context clearly indicates otherwise. For example, the sequence 5′-GAATCC-3′, is understood to include 5′-GAAUCC-3′, 5′-GGATTC-3′, and 5′GGAUUC-3′.

As used herein, the term “recombinant polypeptide” such as Ndfip1 fusion polypeptide, refers to a polypeptide that is produced by recombinant DNA techniques, for example, where a gene encoding a protein or RNA is generally inserted into a vector of recombinant DNA, suitable for expression and which in turn is used to transform a host cell to produce the polypeptide or RNA or where polypeptide is chemically synthesized.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced using recombinant technology, but is not necessarily translated from a designated nucleic acid sequence or polynucleotide. It can be generated in any manner, including by chemical synthesis. A polypeptide also includes a fusion of two or more discrete amino acid sequences.

A “fusion polypeptide” comprises a first amino acid sequence linked to a second amino acid sequence with which it is not naturally linked in nature. The amino acid sequences which normally exist in separate proteins can be brought together in the fusion polypeptide, or the amino acid sequences which normally exist in the same protein can be placed in a new arrangement in the fusion polypeptide, e.g., fusion of a Ndfip1 peptide with a neuron transport moiety. A fusion protein is created, for example, by chemical synthesis, or by creating and translating a polynucleotide in which the peptide regions are encoded in the desired relationship.

“Ndfip1 fusion polypeptide” as used herein refers to a polypeptide comprising a Ndfip1 peptide having at least about 70%, at least about 75%, at least about 80%, at least about 85%, at least about 90% or at least about 95% sequence identity to a polypeptide sequence as shown for example in Ensembl: ENSG00000131507 OMIM: 612050 UniProtKB: Q9BT67, may be encoded by the nucleotide sequence as set forth in for example, Gene Accession Number: 80762 or the codon optimized sequence as set forth in SEQ ID NO: 2 or sequence having at least about 70%, at least about 75%, at least about 80%, at least about 85%, at least about 90% or at least about 95% sequence identity to the Gene Accession Number: 80762 or SEQ ID NO:2, and which maintains the ability to ubiquitinate PTEN; and a neuron transport moiety (also referred to as a neuron specific tag). The Ndfip1 fusion polypeptide may be made as described in the Example 1. The Ndfip1 fusion polypeptide may comprise a human or non-human Ndfip1 peptide, optionally a mammalian Ndfip1 peptide, such as mouse or rat Ndfip1, preferably the human Ndfip1 peptide (for example as shown in SEQ ID NO: 24).

As used herein, the term “injury” or “injuries” includes both traumatic and non-traumatic injury. Examples of non-traumatic injuries include Degenerative Cervical Myelopathy (DCM) and cervical spondylotic myelopathy (CSM).

As used herein, the term “Ndfip1” refers to the NEDD4 family-interacting protein 1, which may be also known as N4WBP5, Putative NF-Kappa-B-Activating Protein 164, Putative NFKB And MAPK-Activating Protein, Breast Cancer-Associated Protein SGA-1M. All Ndfip1 including naturally occurring Ndfip1 may be used. For example, Ndfip1 may be mammalian, for example human Ndfip1, rat Ndfip1 or mouse Ndfip1. The term “Ndfip1 peptide” as used herein can comprise full length Ndfip1 and fragments that can for example induce axonal growth assessed for example in assay as described in the Examples.

The term “neuron transport moiety” as used herein refers to a peptide that can be linked to a cargo and which can permeate a neuron (e.g. for example by receptor mediated internalization), bringing its cargo into the neuron, for example, by binding to a neuron receptor causing it and its cargo along with the receptor to be endocytosed or transported into the neuron. The cargo may be for example the associated Ndfip1 peptide. A neuron-targeting ligand is a type of neuron transport moiety. As used herein, a “neuron-targeting ligand” is a fragment or domain from a neuropeptide, nerve growth factor, or neuron-specific toxin that has the ability to bind a neuron specific receptor and induce endocytosis or transport of the receptor into the neuron. Neuron transport moieties can be linked to a cargo at either or both of the N-terminal or C-terminal end of the cargo. Examples of neuron-transport moieties include for example, a fragment of the translocation domain of diphtheria toxin (DTT) (for example, amino acids 195-388 of Accession Number UniProtKB-P00588 (DTX_CORBE)) and nontoxic C fragment of tetanus toxin (TTC) (for example, amino acids 389-849 of Accession Number UniProtKB-P04958 (TETX_CLOTE)), non-toxic pentameric b chain of the “Cholera toxin” (CTb) from Vibrio cholerae, Rabies Virus glycoprotein (RVG) (having an amino acid sequence of for example YTIWMPENPRPGTPCDIFTNSRGKFRASNG as set forth in SEQ ID NO: 1 or an amino acid sequence with at least about 90% sequence identity to SEQ ID NO: 1), Neurotensin (NT) (having an amino acid sequence of for example LYENKPRRPYIL as set forth in SEQ ID NO: 3 or an amino acid sequence with at least about 90% sequence identity to SEQ ID NO: 3), or Tet1 (having an amino acid sequence of for example HLNILSTLWKYRC as set forth in SEQ ID NO: 4 or an amino acid sequence with at least about 90% sequence identity to SEQ ID NO: 4). For example, in some embodiments, an analog of the above where for example 1, 2 or 3 amino acids may be different, and the neuron transport moiety maintains the ability to facilitate transport a into a neuron.

As used herein, the term “pharmaceutically acceptable carrier” is intended to include any and all solvents, media, isotonic and absorption delaying agents, and the like, compatible with pharmaceutical administration and for use with cells. Optional examples of such carriers or diluents include, but are not limited to, buffered saline, culture media, Hanks' Balanced Salt solution, ringer's solutions, and 5% human serum albumin and bovine serum albumin (BSA). Other carriers may also be used, for example, water may be used with nucleic acid molecule and constructs described herein. The nucleic acid molecules and constructs reconstituted with water or saline may be combined with one or more carriers, for example prior to administration.

The term “neural progenitor cell” also referred interchangeably as neural stem cell (NSC), neural precursor cells (NPC), neural stem progenitor cells (NSPCs) or Neuroectodermal cells (NPCs), as used herein includes neural cells that express Sox2, Pax6 and Nestin and are tripotent and differentiable to neurons, astrocytes or oligodendrocytes.

The term “neural progenitor cell with a spinal cord identity” or “spNPC” refers to neural progenitor cells that can terminally differentiate to spinal cord specific neuronal cell types like ventral motor neurons and spinal interneurons, Renshaw cells, paragriseal, interstitial and propriospinal interneuron cells, and which express elevated levels of spinal cord genes such as Hox genes such as Hox A, B, C or D, 1-10 (e.g. A4, B4, C4) in a higher amount than brain NPCs and express lower amounts of brain markers for example Gbx2, Otx2, FoxG1, Emx2 and/or Irx2 as well as Pax6 as compared to brain NPCs. Methods for producing spNPCs in vitro are provided herein.

The term “oligodendrocyte progenitor cells” or “oNPC” refer to a subtype of glial cells responsible for myelin regeneration. Oligodendrocytes (OLGs) originate from Oligodendrocyte Precursor Cells (OPCs) and are the myelinating cells in the central nervous system (CNS).

The term a “vector” refers to any vehicle for the cloning of and/or transfer of a nucleic acid molecule or expression cassette comprising the nucleic acid molecule into a host cell, for example for expressing the nucleic acid molecule. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. A “vector” includes both viral and nonviral vehicles for introducing a nucleic acid molecule or expression cassette into a cell in vitro, ex vivo or in vivo. A large number of vectors are known and used in the art including, for example, plasmids, modified eukaryotic viruses, or modified bacterial viruses. Insertion of a polynucleotide such as an expression cassette into a suitable vector can be accomplished by ligating the appropriate polynucleotide fragments into a chosen vector that has complementary cohesive termini. The vector comprising the nucleic acid molecule or the expression cassette can be referred to as a vector construct or construct herein. An expression cassette can refer to a coding sequence also referred to as an open reading frame (e.g. a nucleic acid molecule encoding a fusion Ndfip1 polypeptide) and additional sequence optionally to facilitate cloning or expression, such as untranslated sequence, flanking restriction endonuclease site(s) (optionally cut), promoter and/or an integration element.

The term “safe harbor site” includes any genomic location where new genes or genetic elements (e.g., a construct or expression cassette) can be introduced without disrupting the expression or regulation of adjacent genes. Examples include adenovirus associated virus (AAV) integration site, which integrates into the host genome at 19q13.4 qtr (AAV-S1), CCR5integration site and hROSA26 integration site.

The term “construct” as used herein can refer to an expression cassette comprising a Ndfip1 nucleic acid molecule (e.g. encoding a Ndfip1 fusion polynucleotide), or a vector construct wherein the nucleic acid or expression cassette is comprised in a vector such as a viral vector, plasmid, etc.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

A first aspect of the invention includes a Ndfip1 fusion polypeptide comprising a neuron transport moiety and a Ndfip1 peptide. In another embodiment, the neuron transport moiety is or comprises a neuron surface receptor ligand.

In an embodiment, the neuron transport moiety is or comprises an antibody.

Single domain antibody (sdAb), also known as single variable heavy chain (VHH) or single chain antibodies can be used for receptor-mediated transcytosis (RMT) of fusion protein. Several different antibodies for example, sdAb, against different neuron specific surface proteins can be used. Examples include antibodies targeting for transferrin receptor (TfR), insulin receptor (IR), p75-NTR, or GT1b.

For example, the Ndfip1 fusion polypeptide can comprise a neuron transport moiety, an antibody optionally a sdAb, a linker and the Ndfip1 peptide. The neuron transport moiety may be at the N terminus or the C terminus.

In another embodiment the neuron transport moiety comprises a Rabies Virus Glycoprotein (RVG) peptide and has the sequence of YTIWMPENPRPGTPCDIFTNSRGKFRASNG as set forth in SEQ ID NO:1. In another embodiment, the neuron transport moiety is a fusion of a fragment of the translocation domain of diphtheria toxin (DTT) (amino acids 195-388 of Accession Number UniProtKB-P00588 (DTX_CORBE)) and nontoxic C fragment of tetanus toxin (TTC) (amino acids 389-849 of Accession Number UniProtKB-P00588 (DTX_CORBE)), or a non-toxic pentameric b chain of the “Cholera toxin” (CTb) from Vibrio cholerae, or Neurotensin (NT) for example having the sequence of LYENKPRRPYIL as set forth in SEQ ID NO: 3, or Tet1 for example having a sequence of HLNILSTLWKYRC as set forth in SEQ ID NO: 4. In a preferred embodiment, the neuron transport moiety is the fusion of a fragment of the translocation domain of diphtheria toxin (DTT) (amino acids 195-388) and nontoxic C fragment of tetanus toxin (TTC) (amino acids 389-849). In another embodiment, the neuron transport moiety is selected to target motor neurons, and is for example, Tet1.

The neuron transport moiety may be linked directly to the Ndfip1 peptide or via a linker. Accordingly, the Ndfip1 fusion polypeptide may comprise a linker. The linker may be any flexible linker of a length of less than about 30 amino acids, and may for example have the sequence of (GGGGS)₃ (e.g., GGGGSGGGGSGGGGS) as set forth in SEQ ID NO: 5. Other linkers can also be used. Other linkers that can be used include GA₂PA₃PAKQEA₃PAPA₂KAEAPA₃PA₂KA (SEQ ID NO: 25), (EAAAK)n (n=1-3) (SEQ ID NO: 26), A (EAAAK)4ALEA (EAAAK)4A (SEQ ID NO: 27), KESGSVSSEQLAQFRSLD (SEQ ID NO: 28), and EGKSSGSGSESKST (SEQ ID NO: 29).

Another aspect of the invention includes a nucleic acid molecule encoding the Ndfip1 fusion polypeptide described herein. In one embodiment, the nucleic acid molecule comprises the codon optimized nucleotide sequence of SEQ ID NO: 2 encoding a Ndfip1 peptide. In another embodiment, the nucleic acid molecule comprises a nucleotide sequence having at least about 70%, at least about 75%, at least about 80%, at least about 85%, at least about 90%, at least about 95% sequence identity to SEQ ID NO: 2. In another embodiment, the nucleic acid molecule comprises the nucleotide sequence as set forth in Gene Accession Number: 80762 or a sequence having at least about 70%,,at least about 75%, at least about 80%, at least about 85%, at least about 90% or at least about 95% sequence identity to the Gene Accession Number: 80762. In another embodiment, the nucleic acid molecule comprises the sequence of human Ndfip1 (e.g., Gene Accession Number: 80762), rat Ndfip1 (e.g., UniProtKB-Q5U2S1 (NFIP1_RAT); NP_001013077.1, NM_001013059.1 or XP_006254695.1, XM_006254633.3) or mouse Ndfip1 (e.g. UniProtKB-Q8ROW6 (NFIP1_MOUSE); NP_075372.1, NM_022996.1; or XP_006526212.1, XM_006526149.1).

Another aspect of the invention includes a construct comprising the nucleic acid molecule described herein. For example, the construct can comprise or be an expression cassette, the expression cassette including a coding region that encodes a polypeptide, for example a coding region that encodes the Ndfip1 fusion polypeptide, and the construct and/or expression cassette comprising a promoter and/or other transcription or translation control elements operably associated with one or more coding regions e.g., the promoter and/or other transcription or translation control elements may be in the expression cassette or provided by a vector (e.g. a construct comprising the expression cassette. In an operable association, a coding region for a gene product, e.g., a polypeptide, is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed.

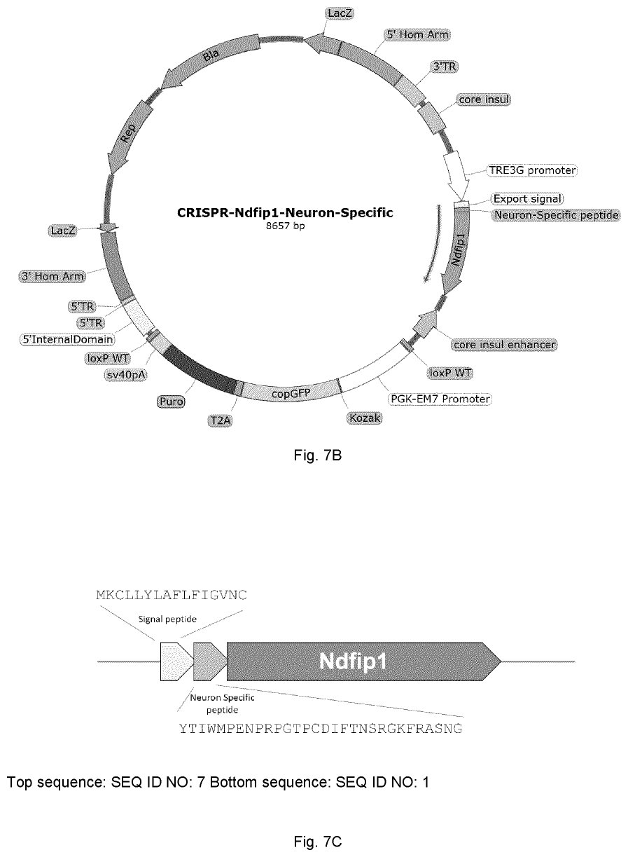

In some embodiments, the construct comprises an expression cassette, and for example comprising one or more of the components or all of the components as illustrated in FIG. 7A.

In another embodiment, the construct comprises 3′ and 5′ homology arms for homologous recombination. CRISPR/Cas9 system for example can be used for the integration of the expression cassette into the host genome. The homology arms can be selected to introduce the expression cassette into a safe harbour site in the genome. In an embodiment, the “safe harbor” site AAV-S1 is used for the integration and the homology comprise sequences that flank the AAV-S1 site. Other safe harbor sites for the integration of the expression cassette can be used.

A wide variety of expression cassette or constructs can be used, either integrating or non integrating, viral or non-viral, epitomal, stabile or non stable. The expression cassette or construct can be a mRNA optionally used alone. For example, the expression cassette and the construct each comprise a nucleic acid encoding the Ndfip1 fusion polypeptide.

Various methods can be used to make constructs for fusion/recombinant proteins, like PCR amplification using specific primers, cutting with restriction enzymes and ligation; homologous recombination strategies like gateway system, Gibson assembly or synthesis of the expression cassette or construct including a vector construct using DNA synthesis.

In other embodiments, the expression cassette may be inserted into a vector (e.g., the construct is a vector construct e.g. a vector comprising the expression cassette), and the vector may comprise various elements, for example comprising one or more of the components or all of the components as shown in FIG. 7B, using the CRISPR/Cas9 vector, PiggyBac vector, or for example, based on viruses, e.g. a viral vector, most notably Herpes Simplex Virus, Adenovirus, Adeno-associated virus (AAV) and retroviruses including lentiviruses. Alternative approaches include the use of naked DNA (e.g., the expression cassette), mRNA, or a construct such as plasmid DNA as well as lipid particle e.g. liposome-nucleic acid molecule complexes.

In an embodiment, the promoter is an inducible promoter. The inducible promoter can be any promoter that is inactive until an inducing agent activates it and initiates transcription, for example any inducible system such as Tet-On, Cumate, Maltose, abasic acid, CRISPR-inducible and the like. In an embodiment, the inducible promoter is the Tet-On inducible promoter TRE3G.

In another embodiment, the construct comprises an expression cassette that further comprises an export signal polynucleotide. The export signal polynucleotide can be any polynucleotide encoding a peptide that signals the secretion of the polypeptide from the cell for example those polynucleotides that encode the peptides listed in Table 1, preferably VSV-G and Human IgG H7.

TABLE 1
Examples of Export Signal Sequences

| Leader/export | |
|---|---|
| sequence Name | Sequence |
| Human OSM | MGVLLTQRTLLSLVLALLFPSMASM as set forth in SEQ ID NO: 6 |
| VSV-G | MKCLLYLAFLFIGVNC as set forth in SEQ ID NO: 7 |
| Mouse Ig Kappa | METDTLLLWVLLLWVPGSTGD as set forth in SEQ ID NO: 8 |
| Human IgG2 H | MGWSCIILFLVATATGVHS as set forth in SEQ ID NO: 9 |
| BM40 | MRAWIFFLLCLAGRALA as set forth in SEQ ID NO: 10 |
| Human IgG H7 | MEFGLSWVFLVALFRGVQC as set forth in SEQ ID NO: 11 |
| Secrecon | MWWRLWWLLLLLLLLWPMVWA as set forth in SEQ ID NO: 12 |
| Human IgKVIII | MDMRVPAQLLGLLLLWLRGARC as set forth in SEQ ID NO: 13 |
| CD33 | MPLLLLLPLLWAGALA as set forth in SEQ ID NO: 14 |
| tPA | MDAMKRGLCCVLLLCGAVFVSPS as set forth in SEQ ID NO: 15 |
| Human | MAFLWLLSCWALLGTTFG as set forth in SEQ ID NO: 16 |
| Chymotrypsinogen | |
| Human trypsinogen-2 | MNLLLILTFVAAAVA as set forth in SEQ ID NO: 17 |
| Human IL-2 | MYRMQLLSCIALSLALVTNS as set forth in SEQ ID NO: 18 |
| Gaussia luc | MGVKVLFALICIAVAEA as set forth in SEQ ID NO: 19 |
| Albumin(HSA) | MKWVTFISLLFSSAYS as set forth in SEQ ID NO: 20 |
| Influenza | MKTIIALSYIFCLVLG as set forth in SEQ ID NO: 21 |
| Haemagglutinin | |
| Human insulin | MALWMRLLPLLALLALWGPDPAAA as set forth in SEQ ID NO: 22 |
| Silkworm Fibroin LC | MKPIFLVLLVVTSAYA as set forth in SEQ ID NO: 23 |

Export sequences (also referred to as signal sequences) are typically upstream (e.g., 5′) and fused in frame or operatively linked in the construct to the encoded polypeptide they are meant to usher out of the cell. For example, the export sequence can be upstream of the neural transport moiety which is upstream of the Ndfip1 peptide.

Another aspect of the invention includes a cell expressing the Ndfip1 fusion polypeptide described herein, wherein the cell comprises the construct described herein for expressing and secreting the Ndfip1 fusion polypeptide. In an embodiment, the cell is a human cell. In one embodiment, the cell comprises a construct comprising an inducible promoter and/or an export signal polynucleotide. The inducible promoter and/or signal sequence are operatively linked to the nucleic acid molecule encoding the Ndfip1 fusion polypeptide. In one embodiment, the cell is a neural progenitor cell (NPC). In another embodiment, the NPC is an oligodendrogenic NPC (ONPC). In another embodiment, the NPC is a spinal identity NPC (spNPC). In a further embodiment, the cell is a fibroblast. Fibroblast cells can be used for example to make induced pluripotent cells (iPSCs). iPSCs can be used to prepare NPCs. In one embodiment, the NPC are made using the methods described in Examples 1 and 2 or any other method in the art for producing NPC, for example those found in Khazaei, Mohamad et al. “Generation of Definitive Neural Progenitor Cells from Human Pluripotent Stem Cells for Transplantation into Spinal Cord Injury.” Methods in molecular biology (Clifton, N.J.) vol. 1919 (2019): 25-41, which is hereby incorporated by reference.

The cells can be differentiated to for example NPCs before or after introducing a nucleic acid, expression cassette or construct for producing the Ndfip1 fusion polypeptide.

In another embodiment, the cell described herein secretes the Ndfip1 fusion polypeptide at a concentration of about 3 ng/ul to about 50 ng/ul, optionally, about 3 ng/ul, about 6 ng/ul, about 12 ng/ul, about 25 ng/ul or about 50 ng/ul, preferably about 12 ng/ul.

In an embodiment, the cell secretes the Ndfip1 fusion polypeptide at a concentration of about 12 ng/ul.

Another aspect of the invention includes a therapeutic for use or method for treating a neurodegenerative disease and/or an optic nerve, brain, and/or spinal cord injury comprising use of or administering the Ndfip1 fusion polypeptide described herein, a Ndfip1 nucleic acid molecule, construct or cell expressing the Ndfip1 fusion polypeptide described herein. The polypeptide, nucleic acid molecule, construct or cell is administered to a subject in need thereof. In an embodiment, the therapeutic is a cell described herein. In embodiments comprising a cell, nucleic acid molecule and/or construct, an inducible promoter and/or encoded export sequence may be present so that the fusion polypeptide is secreted from the cell, for example upon induction. In various embodiments, an inducible promoter and nucleic acid encoding an export sequence operatively linked to the nucleic acid molecule encoding the Ndfip1 fusion polypeptide are present.

Another aspect of the invention includes a method of treating a neurodegenerative disease and/or an optic nerve, brain, and/or spinal cord injury in a subject in need thereof, the method comprising:
a. administering the Ndfip1 fusion polypeptide, nucleic acid molecule or construct described herein to the subject; or
b. administering the cell described herein to the subject followed by an inducing agent, wherein the cell comprises a construct the construct comprising an inducible promoter and/or signal sequence operatively linked to the nucleic acid molecule encoding the Ndfip1 fusion polypeptide.

In an embodiment, the Ndfip1 fusion polypeptide, nucleic acid molecule, expression cassette, construct, composition described herein or the cell is administered to or proximal to neurons damaged by the neurodegenerative disease and/or the optic nerve, brain, and/or spinal cord injury. For example, the administration can be to the motor neurons where the neurodegenerative disease affects the motor neurons, as for example in ALS.

The cell may be in a cell suspension e.g. a composition comprising the cell and a pharmaceutically acceptable carrier. The cells may be suitably prepared for administration according to the disease or condition to be treated. For example,, the cell suspension can be injected at or proximal to a site of injury using for example a with special needle and syringe without surgery. Alternatively, the cells, compositions, nucleic acid molecules and Ndfip1 fusion polypeptides may be administered during surgery. for example, in cases of spinal cord injury.

In some embodiments, the subject in need thereof has a neurodegenerative disease. In some embodiment, the subject in need thereof has an optic nerve, brain and/or spinal cord injury. In some embodiments, the subject in need thereof has a spinal cord injury.

In the case of spinal cord injury for example the cell administered may be a cell described herein, for example a spNPC modified to express the Ndfip1 fusion polypeptide, optionally when induced.

In another embodiment, the method comprises administering the cell described herein to the subject followed by an inducing agent. The inducing agent can be any agent capable of activating an inducible promoter so that for example, transcription of a gene may be initiated. For example, wherein the inducible promoter is responsive to tetracycline, cumate, maltose or abasic acid responsive, tetracycline, cumate, maltose, abasic acid or a corresponding analog thereof (e.g., doxycycline is a corresponding analog of tetracycline) can be administered, respectively

The sequences of various inducible promoters are known. For example, the Tet-ON sequences are available in GenBank: MK816964.1, as is the cumate promoter e.g., GenBank: KF536588.1). In another example, the inducible promoter can be a CRISPR-inducible promoter using a method described in the art.^{36,37} In one embodiment, the inducible promoter is TRE3G and the inducing agent is doxycycline.

In another embodiment, the treatment is for the neurodegenerative disease. In another embodiment, the neurodegenerative disease is multiple sclerosis (MS), amyotrophic sclerosis (ALS), Alzheimer's disease, Parkinson's Disease, or Huntington's Disease.

The kind of cell used in the method of treatment may be selected based on the disease, for example where the neurodegenerative disease is biased towards affecting a subset of neurons, for example, where the neurodegenerative disease is ALS and the damage is biased towards motor neurons or in MS where oNPCs may be more useful in treatment given that it is a myelination disease. The treatment can be started as soon as possible for example to reduce further damage. For traumatic injuries, it may be beneficial to start treatment as soon as inflammation is reduced. The duration of treatment depends on the neurological recovery. For example, he induction of the expression can be stopped (e.g., administration of the inducing agent can be stopped) when the neurological recovery plateaus.

In an embodiment, the treatment is for the optic nerve, brain, and/or spinal cord injury. In another embodiment, the treatment is for the spinal cord injury. In another embodiment, the Ndfip1 fusion polypeptide or the cell is administered to the subject not earlier than two weeks following the optic nerve, brain and/or spinal cord injury.

In another embodiment, the subject is a human.

Another aspect of the invention includes a method of making the cell described herein, the method comprising the following steps:
a. preparing an expression cassette comprising a nucleic acid molecule encoding a Ndfip1 fusion polypeptide described herein operatively linked to a promoter, optionally wherein the promoter is an inducible promoter;
b. inserting the expression cassette described herein into a vector to produce a vector construct; and
c. introducing the vector construct into a cell.
d. selecting the cell expressing or capable of expressing (e.g., when administered inducing agent) the Ndfip1 fusion polypeptide.

The vector can be a vector described herein and the expression cassette and/or vector construct can comprise an inducible promoter and/or an export sequence so that the protein may be inducible and/or secreted.

The cell can be a cell described herein. For example, the cell may be a NPC, optionally with spinal identity (spNPC) or an oligodendrogenic NPC (ONPC). The cell may be any neural stem/progenitor cell, progenitor of motor-neurons, differentiated neurons, neural stem cell, ventral neural progenitor cell, motor neuron progenitor (MNP), Motor Neural Progenitor Cell (pMN), Neuroepithelial precursor cell, or any of the central nervous system (CNS) neuronal cell types.

The spNPC can be prepared as described herein. For example the method may comprise one or more steps described in Example in PCT application PCT/CA2021051239 filed Sep. 8, 2021, titled METHODS FOR GENERATING NEURAL PROGENITOR CELLS WITH A SPINAL CORD IDENTITY, herein incorporated by reference. The nucleic acid molecule, expression cassette or construct for expressing the Ndfip1 fusion protein, can be introduced prior to differentiating or after differentiating the cells to spNPC or further differentiated lineage cells.

The cell may also be made using any applicable methods known in the art for making a cell expressing a Ndfip1 fusion polypeptide, for example, transfection for example with polyethylenimine, electroporation, microinjection, transduction, cell fusion, DEAE dextran, calcium phosphate precipitation, lipofection (lysosome fusion), use of a gene gun, or a DNA vector transporter. The vector may be for example, a PiggyBac vector, or for example, based on viruses, most notably Herpes Simplex Virus, Adenovirus, Adeno-associated virus (AAV) and retroviruses including lentiviruses.

Another aspect of the invention includes a composition comprising the Ndfip1 fusion polypeptide described herein, the nucleic acid molecule, the construct or the cell described herein and optionally a pharmaceutically acceptable carrier.

In one embodiment, the composition comprises the Ndfip1 fusion polypeptide combined with a therapeutically suitable hydrogel that slowly releases the polypeptide. The composition comprising the hydrogel could for example be administered via injection of the hydrogel intrathecaly in the spinal cord. In another embodiment, an osmotic pump filled with the Ndfip1 fusion polypeptide, can be used to supply a catheter, where the catheter is placed in or close to the injury site, for example in a brain injury or for treating a neurodegenerative disease affecting the brain, the catheter can be put under the dura to slowly release the Ndfip1 fusion polypeptide. In another embodiment, the Ndfip1 fusion polypeptide may be administered via injection of AAV viruses that can express the Ndfip1 fusion polypeptide.

Another aspect of the invention is a use of a Ndfip1 fusion polypeptide, a nucleic acid molecule, a construct or a cell inducibly expressing and secreting the Ndfip1 fusion polypeptide in the manufacture of a medicament for treating a neurodegenerative disease and/or an optic nerve, brain, and/or spinal cord injury.

Another aspect of the invention is use of a Ndfip1 fusion polypeptide, a nucleic acid, a construct or a cell inducibly expressing and secreting the Ndfip1 fusion polypeptide to treating a neurodegenerative disease and/or an optic nerve, brain, and/or spinal cord injury.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1: Overexpression of Ndfip1 in Cells

### Neuronal Cultures and Transfection

For cultures of dissociated pyramidal neurons, the hippocampus from E18 rat embryos were dissected and dissociated, and neurons were plated onto glass coverslips coated with laminin and Poly L lysine (PLL) at a density of 800,000 cells/coverslip in 24-well plates. Neurons were transfected at the time of plating with Ndfip1 or Nedd4 expressing plasmids using the Amaxa™ nucleofection method. Neurons were fixed at 3 days in vitro (div) with 4% paraformaldehyde and 15% sucrose in phosphate-buffered saline for 20 min at 4° C.

### In Vitro Model of Axon Injury:

Neurons were grown on BioFlex® six-well plates and were submitted to mechanical stretch to apply a strain to cells cultured on elastic silicone membranes. Cells were subjected to an equibiaxial static strain of 30% for 1 h. Cells were then incubated for another 3 days in vitro and then used for a TUNEL assay, western blotting, and immune staining. At acute and subacute stages post-stretch (1 and 24 h post-stretch, respectively) cell viability was investigated using propidium iodide (PI).

### Neurite Outgrowth Assay

Neurons were grown for approximately 3 days post injury and then fixed in 4% PFA/20% sucrose in PBS, stained with anti-βIII tubulin antibody (manufactured by Covance) and an anti-mouse-FITC secondary antibody (Invitrogen). Neurite outgrowth length and the number of neurons were analyzed with using ImageJ software.

Generation of NPCs from hiPSCs

The hiPSC lines were differentiated to NPCs using dual SMAD inhibition in monolayer culture. At the start of differentiation (day 0), hiPSCs were dissociated to single cells and re-plated as a monolayer on Matrigel (Corning, Tewksbury, MA) with a density of 20,000 cells/cm² in mTeSR1 media. After cells reached 90% confluency, media was gradually changed over two days to neural induction media (NIM), consisting of a 1:1 ratio of DMEM: F12 media supplemented with B27, N2, FGF (10 ng/ml), 10 μM TGFβ inhibitor (SB431542), 200 ng/ml Noggin and 3 μM GSK3β inhibitor (CHIR99021). After 7 days in culture, the neural rosettes were manually isolated and plated as single cells on poly-L-lysine (PLL)/Laminin coated dishes in NPC expansion media (NEM), consisting of neurobasal media supplemented with B27, N2, FGF (10 ng/ml) and EGF (20 ng/ml) for two passages. The resulting cells were then cultured in NEM as single cells on Ultra-Low adherent dishes (Corning, Tewksbury, MA) at a density of 10,000 cells/ml to form primary neurospheres. After 5 days in culture, each individual clonal neurosphere was separately plated in a well of a PLL/Laminin coated 24 well plate to proliferate. The steps were then repeated to get the secondary clonal neurospheres. For expansion of the culture, secondary clonal neurospheres were cultured in NEM on PLL/Laminin. During the period of induction, which took over 2 weeks, the cells progressed through the neural rosette and neurosphere stages.

### Genetic Modification of NPCs

Human NPCs were stably transfected with a piggyBac vector to express TAT-Ndfip1 or neuron transport moiety -Ndfip1. For the construction of piggyBac-Ndfip1, a codon optimized variant of the human Ndfip1 gene was custom synthesized and inserted into the BsaI site of the piggyBac vector. The piggyBac vector carried an ires-GFP downstream of the cloning site. Cells were transfected using Amaxa™ Nucleofection kit for neural stem cells (Lonza) according to the manufacturer's protocol. Single cell fluorescence-activated cell sorting (FACS) for GFP signals was used to establish clonal lines.

In vitro differentiation and immunocytochemistry:

In order to examine the differentiation potential of the hiPSC-NPCs and analyze whether there were any differences between Ndfip1 treated NPCs vs. control NPCs, cells were differentiated in vitro by culturing them in pro-neurogenic, -oligodendrogenic and -astrocytogenic conditions. For the neurogenic condition, cells were cultured for two weeks on PLL/laminin substrate in a neurobasal medium supplemented with B27, N2, Retinoic Acid (0.1 μM), cAMP (100 ng/ml), and brain-derived neurotrophic factor (BDNF, 10 ng/ml; PeproTech, Rocky Hill, NJ) in the absence of FGF and EGF. To induce astrocyte differentiation, hiPSC-NPCs were cultured on Matrigel in DMEM/F12 supplemented with B27, 0.1% fetal bovine serum (FBS), BMP4 (10 ng/ml, Peprotech) and CNTF (5 ng/ml; PeproTech) for 14 days. To promote oligodendrocyte differentiation, hiPSC-NPCs were cultured on Matrigel in DMEM supplemented with N2 supplement, and treated for 3 days with Retinoic Acid (0.1 μM). The Shh agonist, Purmorphamine (1 μM) was added from day 2 for 7 days. On day 7, PDGF-AA (20 ng/ml) was added for another 7 days. To enhance the maturation of oligodendrocytes, triiodothyronine (T3) (30 ng/ml; Sigma-Aldrich) was added during the final phase of differentiation for 6 days. Morphological analyses and immunostaining with markers for neurons and astrocytes were performed after 14 days in vitro differentiation and after 21 days for oligodendrocytes.

### Quantitation of Secreted Ndfip1 Using Sandwich ELISA

The supernatants of medium were collected and a protease inhibitor cocktail (2.5 mM EDTA, 10 μM leupeptin, 1 μM peptastin and 1 mM phenylmethylsulfonyl fluoride) was added. Ndfip1 level was assayed with a sandwich ELISA. Colorimetric Immunoassay protocol. Monoclonal anti-Ndfip1 (abcam) was used in twofold serial dilutions starting at 10 ng ml-1. Flat-bottom, 96-well plate (Nunc) was coated with Ndfip1 antibody overnight at 1:250 dilution. Plates were blocked with 10% FCS in PBS buffer for 2 h and incubated with sample condition media containing secreted Ndfip1 for 2 h at room temperature and detected with HRP-conjugated Ig subclass antibody for 1 h at room temperature. Plates were developed with TMB substrate solution and read at 450 nM using a microplate reader (TECAN).

### TUNEL Assay:

For the measurement of apoptosis, DNA fragmentation was investigated using the in situ colorimetric TUNEL assay according to the manufacturer's instructions. Briefly, cells were fixed with 3.7% buffered formaldehyde solution for 5 min and washed with PBS. Cells were then permeabilized with 100% methanol and digested with proteinase K for 15 min. Then cells were labeled and incubated with deoxynucleotidyl transferase at 37° C. for 90 min. The cells were then incubated with Sapphire substrate for 30 min. The colorimetric reaction was stopped with 0.2 N HCl and measured in a microplate reader at 450 nm absorbance.

qRT-PCR:

Quantitative RT-PCR (q-RT-PCR) was used to examine the expression profile of differentiation markers in cells. For characterization of Ndfip1 treated hiPSC-NPCs, neural, astrocytic and oligodendroglial markers were examined with the use of appropriate primers. mRNA was isolated using the RNAeasy mini kit (Qiagen, Hilden, Germany). A NanoDrop™ spectrophotometer was used to evaluate the concentration and purity of the mRNA. cDNA was synthesized using SuperScript® VILO cDNA Synthesis Kit (Life Technologies, Carlsbad, CA) with random hexamere primers according to manufacturer instructions. RT-PCR was performed using TaqMan™ design primers with FAST TaqMan master mix under recommended thermocycling parameters on a 7900HT Real time PCR system. Samples were run in triplicate. Values were normalized to the GAPDH housekeeping gene. For examination of the neural progenitor, neuronal, astrocytic and oligodendroglial markers, results were normalized to GAPDH and to the hiPSC source. Gene expression levels were compared using the 2-^{ΔΔCT} method.

### Results

The effect of Ndfip1 on axon outgrowth and neuronal survival in vitro was investigated.

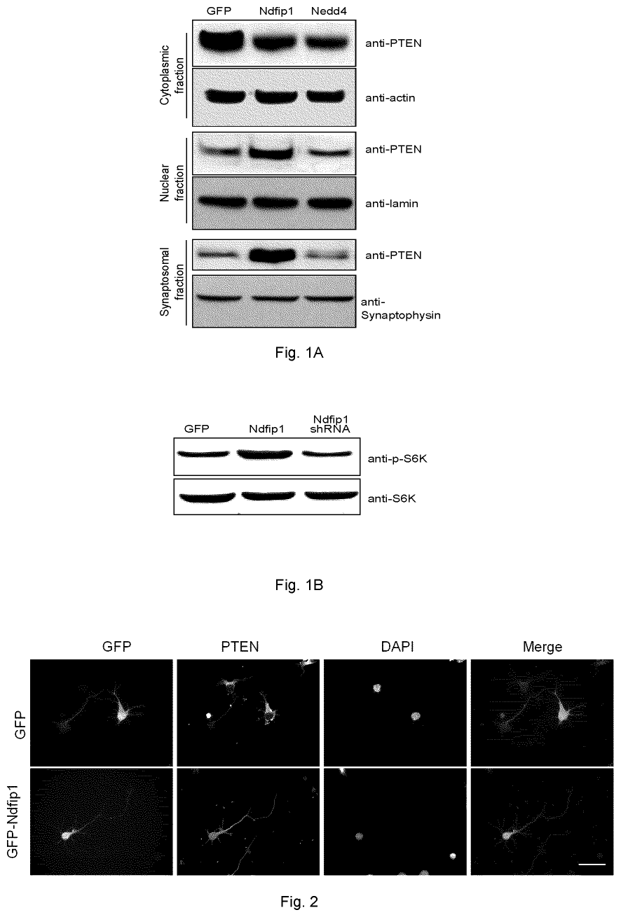

Nedd4 expression in neurons regulates PTEN degradation in cytosol and its trafficking to nucleus and synaptic terminals. Nuclear trafficking of PTEN in neurons is stimulated by overexpression of Ndfip1 in cultured cortical cells (FIG. 1A and FIG. 2). In this experiment, overexpression of Ndfip1 showed much more robust activity than overexpression of Nedd4. Moreover, Ndfip1 induced reduction of PTEN in the cytosol, results in activation of mTOR pathway as assessed by the amount of phospho S6K (FIG. 1B). Down-regulation of Ndfip1 expression in neurons did not have significant effect on mTOR activity.

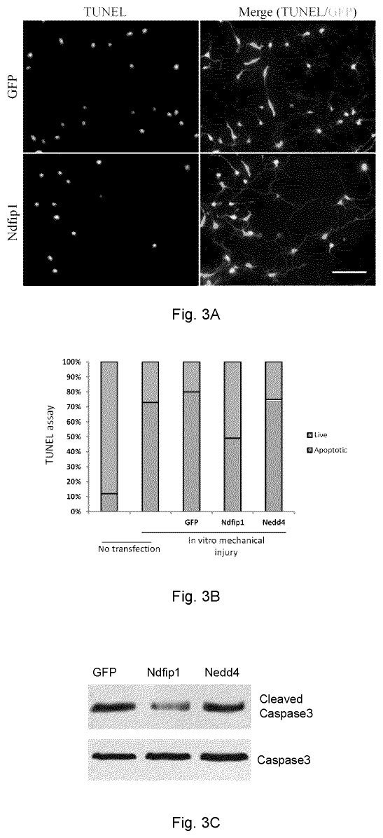

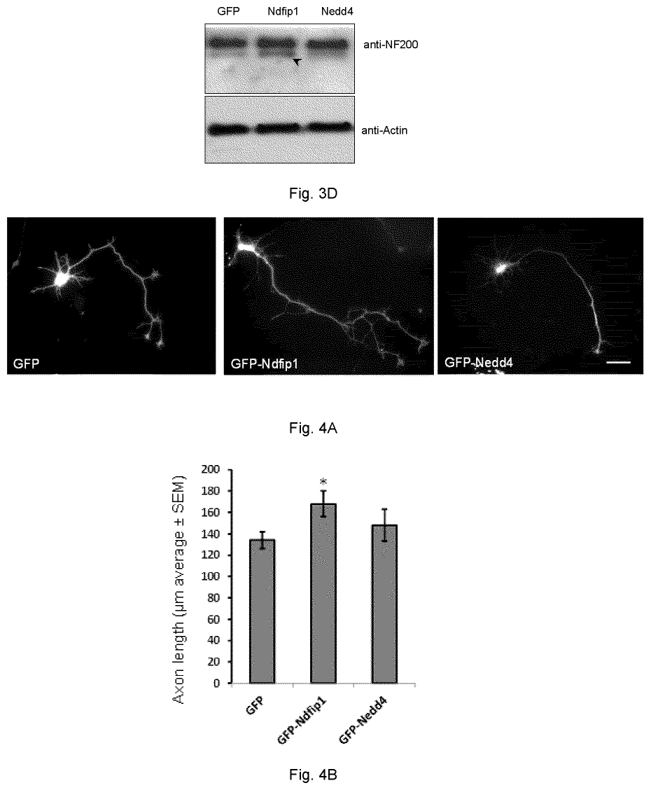

Ndfip1 overexpression increases neuronal survival. To express exogenous proteins in cultured neurons, Nucleofector™ method was used to transfer expression vectors into the cells. Using an in vitro model of axonal injury, around 61%±2% increase in the apoptotic death of the cultured cells was induced, as assessed by TUNEL assay (FIG. 3B). However, neurons transfected with expression vectors for Ndfip1 showed an increased survival (31%±5%) compared to control neurons expressing GFP (FIGS. 3A and B). Furthermore, Ndfip1 expression could reduce cleavage of caspase-3 (FIG. 3C) and also could inhibit degradation of dephosphorylated NF200 (FIG. 3D; arrowhead). Dephosphorylated NF200 was degraded after inducing apoptosis^{28,29}.

Ndfip1 can promote axonal outgrowth. To investigate the function of Ndfip1 in axonal growth, we assessed its influence on axon outgrowth in a cortical culture. Cortical neurons were transfected with an expression vector for GFP, GFP-Ndfip1 or GFP-Nedd4. After 3 days in vitro, neurons were fixed and stained and the axon length of GFP positive neurons were measured. It was found that over-expression of Ndfip1, resulted in the formation of longer axons compared to control neurons (FIGS. 4A and 4B). Over-expression of Nedd4 did not have significant effect on axonal length.

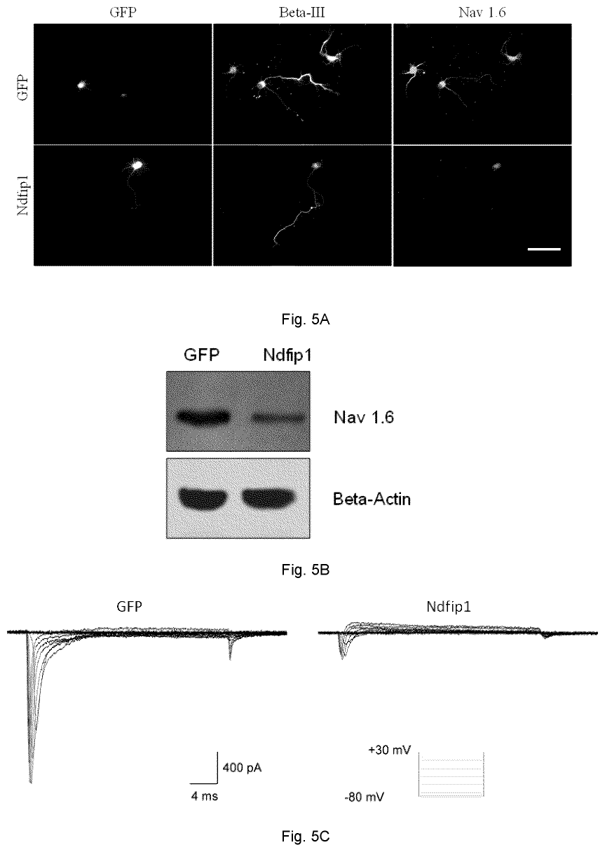

Ndfip1 expression reduces the density of voltage gated sodium channels on axons. It has been shown that Nedd4-Ndfip1 system, robustly ubiquitinate and downregulate voltage sensitive sodium channels^{30,31}. Previous studies has shown that influx of Na⁺ into the cells is an early event in the pathogenesis of secondary traumatic CNS injury³². Without wishing to be bound to theory, Ndfip1 might provide potential neuroprotection effect the same as voltage-gated sodium channel blockers³³. To investigate the effect of Ndfip1 on activity of voltage-gated sodium channels, Ndfip1 was over-expressed endogenously in cultured neurons. FIG. 5A illustrates cortical neurons that were transfected with an expression vector for GFP or GFP-Ndfip1. After 3 days in vitro, neurons were fixed and stained with antibodies against Nav1.6 and Beta-iii tubulin. FIG. 5B depicts the results of a Western blot analysis of lysates of Nav1.6 level from cultured neurons transfected with GFP or Ndfip. FIG. 5C depicts representative current traces showing the effects of Ndfip1 on Na+ currents. Neurons were held at −70 mV and depolarized to voltages of between −50 and +50 mV to evoke the inward Na+ currents. Ndfip1 overexpression resulted in the reduction of Nav1.6 in neurons (FIGS. 5A and B). Ndfip1 overexpression also resulted in the reduction of Na+ currents (FIG. 5C).

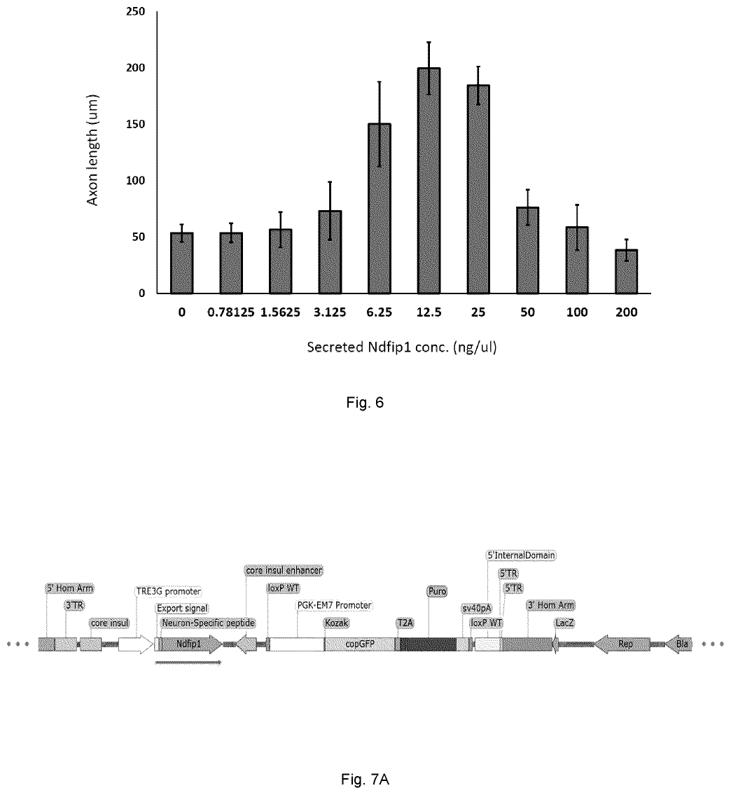

Ndfip1 can also be overexpressed in neurons using inducible cells for inducibly expressing and secreting Ndfip1 into neurons. Cultured neurons were treated after in vitro injury with different concentrations of Ndfip1 than is expressed and secreted by human inducible pluripotent stem cell neural progenitor cells (hiPSC-NPCs). Axon outgrowth was measured after 3 days in vitro (FIG. 6). The most optimal in vitro concentration of Ndfip1 was shown to be around 12 ng/ul (FIG. 6). Where the inducible cells are NPCs, the methods provided in this Example or in Example 2 may be used to make the NPCs.

### Example 2: Method of Making NPCs

An example of a step-by-step protocol for the generation of hPSC-NPCs with a spinal identity (spNPCs) starting from hPSCs is provided herein. The three main steps for the procedure: 1) Generation of unpatterned NPCs or embryoid body (EB) formation and dual-SMAD inhibition 2) Priming NPCs to ectodermal cell fate, and 3) Patterning of NPCs into spNPCs (FIG. 3).

Step 1: Generation of Unpatterned NPCs from hPSCs

Different methods of generating NPCs in vitro, including using “default pathway”^{22,23}, or via inhibition of SMAD signaling pathway. There are protocols utilising inhibition of just SMAD1 by using a BMP inhibitor, or utilizing dual-SMAD inhibitors, to inhibit both SMAD1 and SMAD2 (inhibiting TGFβ). The NPCs that are generated with these protocols in vitro, first acquire rostral identity by default¹⁴, before they get patterned to get other identities.

We use EB culture with dual-SMAD inhibition to generate NPCs with rostral identity. These cells are referred to herein as unpatterned NPCs.

If the hPSCs are cultured on a fibroblast feeder layer, they can be further expanded in feeder-free conditions for 3-4 passages prior to induction of neural progenitors. This action acclimates the cells, improving culture quality and yield.

To generate EBs, small clumps of hPSC will be cultured on ultra-low adherent dishes in hPSC culture media (without FGF2) and neural induction media for 7 days. During this period, hPSCs grow to cell aggregates which are called EBs.

Neuroectodermal induction begins when EBs are transferred into the Neural Induction Medium (NIM) (around day 4-5). Plating EBs on Matrigel or Geltrex in NIM promotes the transition of cells into the rosettes with a neuroectodermal lineage that are expressing Sox1. Sox2 is also in hPSCs, but Sox1 starts after cells get neuroectodermal fate.

FGF2 signaling is necessary for the polarization of rosettes. Fibroblast growth factor 2 (FGF2) is then added to guide the transition of the neuroectodermal cells into rosette structures.

NEM is for transitioning NPCs to produce NPC that express Nestin, Sox2, and Pax6 (e.g., unpatterned NPCs).

### Protocol:

1) Apply Accutase or ReLeSR (Stem Cell Technologies) as per manufacturer instructions to a healthy and homogeneous culture of hPSCs to separate them into cell clumps (consisting of 20-50 cells). Suspend the cells at a density of 1×10⁴ clumps/mL in hPSC culture media and add 2 mL to ultra-low adherent 6-well dishes. Incubate for two days under standard culture conditions of 37° C. and 5% CO₂ in a humidified incubator.

hPSC culture media (Table 2) without FGF is recommended.

TABLE 2
Composition of culture media

| | |
|---|---|
| Component | amount |

hPSC culture media

| DMEM/F-12 Medium | | |
|---|---|---|
| Knockout Serum Replacement (KOSR) | 20% | |
| L-Glutamine | 2 | mM |
| MEM Non-Essential Amino Acids | 1% | |
| FGF2* | 10 | ng/ml |
| 2-Mercaptoethanol | 0.1 | mM |
| Transferrin | 10 | μg/ml |
| IGF-I | 200 | ng/ml |

*at some steps FGF is not used
Neural Induction media (NIM)

| DMEM/F-12 and Neurobasal Media 1:1 | | |
|---|---|---|
| (supplemented with sodium pyruvate) | | |
| B27 minus vitamin A | 1x | |
| N2 supplement | 1x | |
| MEM Non-Essential Amino Acids | 1% | |
| FGF2* | 10 | ng/mL |
| heparin | 1.25 | U/L |
| TGFβ-inhibitor (SB 431542), | 10 | μM |
| BMP-inhibitor (Dorsomorphin) | 2 | μM |

*at some steps FGF is not used
Neural Expansion media (NEM)

| DMEM/F-12 and Neurobasal Media 1:1 | | |
|---|---|---|
| (supplemented with sodium pyruvate) | | |
| B27 minus vitamin A | 1x | |
| N2 supplement | 1x | |
| MEM Non-Essential Amino Acids | 1% | |
| FGF2 | 10 | ng/mL |
| EGF | 10 | ng/mL |
| heparin | 1.25 | U/L |

Alternate methods of passaging to Accutase dissociation include using 0.5 mM EDTA in Dulbecco's PBS without MgCl₂, CaCl₂, or ReLeSR. ReLeSR selectively lifts only iPSC cells, leaving differentiated cells on the plate. This allows for quick and easy selection for regular iPSC culture as well.
2) On day two, gently tilt plates to allow cell clumps to collect in the bottom corner of the well and replace half of the media from the top with fresh hPSC media supplemented with SMAD1 inhibitor Dorsomorphin (2 μM) and SMAD2 inhibitor SB431542 (10 μM). Repeat this process on day 4.

Dorsomorphin and SB431532 block the BMP and TGF-β signaling pathways, which has been shown to improve the efficiency of neural induction to greater than 80% of total cells¹⁴.
3) On day 5, gently tilt plates and replace the media with NIM without FGF2 (Table 2).

Cell aggregates in the form of EBs should be observed by day 5. EBs simulate the endogenous conditions under which pluripotent hPSCs transition into neuroectodermal cells.
4) On day 7, transfer the EBs in NIM with FGF2 to a standard 6 cm culture plate pre-coated for 1 hour at 37° C. with Matrigel or Geltrex. Wait 24 hours before performing microscopy to confirm that EBs have completely settled and adhered to the plates.
5) On day 8, replace half of the media with fresh NIM with FGF2, but without Dorsomorphin. Repeat this process daily until day 13 to 17 (varies based on PSC cell line), at which point the first neural rosettes will form and then neural tube-like structures should be observed. The cells in rosettes or neural tube-like structures, unlike those in the periphery, should express early neuroectodermal markers such as Pax6 and Sox1.
6) Two days after visualizing neural rosettes or neural tube-like structures, use a fine pipette tip to lift and transfer rosettes to a 15 mL Falcon tube containing NEM. Make sure to leave the non-neural cells at the periphery of the plate, as improper selection will impair NPC purity (FIG. 4).

Alternatively, one can use either Neural Rosette Selection Reagent (Stem Cell Technologies), or a brief incubation (3-5 min) with Dispase, tapping, and a PBS wash to lift neural rosettes. Neural Rosette Selection Reagent had been found to be sub-optimal for selectively lifting neural rosettes of monolayer differentiation cultures, so use in only EB cultures is recommended.
7) Resuspend the selected rosettes at 1×10⁵ cells/mL in NEM and plate the cells onto PLL (0.1 mg/ml solution) and laminin pre-coated plates at 1×10⁵ cells/cm². Avoid re-plating at lower densities, as this promotes undesired differentiation and loss of secondary rosette formation.

Laminin-511 (but not -332,-111, or -411) is preferred over other ECM replacements, such as Matrigel or Geltrex due to it being growth-factor free, which may interfere with the differentiation process.

After 7-8 days, lift the secondary neural rosettes manually (or with Dispase), and transfer them to another Matrigel-coated plate with N2B27 media. Following that, dissect the tertiary rosettes to purify NPCs.

Following re-plating, the culture should contain isolated NPCs that express Nestin, Pax6, and Sox2, but not Oct4.

### Clonal Expansion Neurosphere (Primary, Secondary, Tertiary).

8) Culture cells at 10 cells/μL on ultra-low adherent plates in NEM. Tilt plates and replace half the media with fresh media every 2-3 days for one week. At this stage, primary neurospheres should be observed as perfect spherical clusters of cells with a smooth contour and be at least 50 μm up to 150 μm in size. Neurospheres should not appear dark and ragged nor contain vacuoles or dead cells. They may also express Oct4, the marker for primitive NPCs.
9) Following detection, transfer neurospheres to 15 mL Falcon tubes with 500 μL of NEM. Use a flame-polished Pasteur pipette to pipette the media up and down 10-20 times, or until separation into single cells is observed. Plate the suspension at 10 cells/μL on ultra-low adherent plates in NEM.

### Expansion of Cells

10) Culture single cells at 1×10⁴ cells/cm² on PLL/laminin pre-coated standard culture plates in NEM containing 10 μM ROCK inhibitor. The next day, replace with fresh media containing NEM only.
11) After 5-6 days, use Accutase to passage cells to new PLL/laminin pre-coated plates with NEM. One day after passaging, supplement with 10 μM ROCK inhibitor.

Note: hPSC-NPCs generated using this method will, by default, express FoxG1, Gbx2 and Otx2, markers of forebrain to midbrain identity. Cells will not express HoxC4, a marker of spinal identity in NPCs.

### Step 2: Keeping the NPCs in the Ectodermal Cell Fate

During Step 1, Bone Morphogenetic Protein 4 (BMP4) signaling was inhibited by BMP inhibitor Dorsomorphin, but LDN193189 (LDN) or Noggin can also be used, and TGFβ was inhibited by SB431542 (SB) to prevent mesodermal and endodermal differentiation. In the next step (Step 3) we are going to use Retinoic Acid (RA). RA tends to deviate differentiation of cells to a mesodermal fate²⁶.

To keep cells in the ectodermal fate, we need to inhibit Notch signaling when RA is active²⁷. It has been shown that inhibition of Notch signaling inhibits the differentiation to mesodermal fate and keep cells in the ectodermal layer²⁸. To inhibit notch signaling, we use the Notch antagonist EGF-L7 (10 ng/ml). EGF-L7 interacts with all the four Notch receptors (Notch1-4) and inhibits/competes with Jagged1 and Jagged2 proteins (not DLL4) for their interaction with Notch receptors²⁹. EGF-L7 knockdown stimulates the Notch pathway and EGF-L7 over-expression inhibits the Notch pathway. While NPCs are actively proliferating, Notch signaling contributes to the maintenance of the undifferentiated state.

Furthermore, by replacing the EGF in culture media with 10 ng/ml EGF-L7 in this step, EGF-L7 activates EGF-receptor, but it is less potent than EGF and modulates Notch signaling which reduce the hyper-proliferation of NPCs. Optionally we can also add 0.5 μM of DLL4 (DLL4: Delta-Like 4; a Notch agonist) with EGF-L7 to balance the reduction in Notch activity and keep the level of expression of neural progenitor genes like Nestin and Pax6 (FIG. 5).

There are some evidences that during development, unlike anterior neural progenitors, spinal progenitors can be also originated from neuromesodermal progenitors (NMPs). NMPs are able to differentiate into both paraxial mesodermal tissue and posterior neural tissue in vitro, and even further into specific neuron subpopulations such as motor neurons^{30, 31}. In vivo experiments in zebrafish have found that subpopulations of NMPs become fate restricted and spatially segregated, as well as having large differences in self-renewal potential³².

### Step 3: Patterning NPCs Towards a Spinal Cord-Specific Identity:

To generate spNPCs, cells were patterned using a stepwise treatment of morphogens³⁸
1) Dissociate cells into single cells using for example Accutase or other cell detachment solution. Plate cells for example at a density of 1×10⁴ cells/cm² on for example PLL/laminin pre-coated standard culture plates in media such as DMEM: F12 media supplemented with B27, N2, FGF2 (40 ng/ml), FGF8 (200 ng/ml). Incubate under standard conditions for three days³⁸.

Table 3 contains a list of reagents that can be used for this protocol.

In this step a high concentration of FGF2 (from 50 ng/ml up to 150 ng/ml) and a high concentration of FGF8 (from 50 ng/ml to 400 ng/ml) is being used. In the embryo, caudal cells are exposed to select FGFs for longer periods of time than rostral cells they are involved in regionalization of the spinal cord along the rostral-caudal axis. During later stages of spinal cord elongation, FGF8 is more broadly expressed. Expression of FGF8 continues for several days but declines toward the final stages of somitogenesis and the cessation of axis elongation^{39,40}. Treatment with FGF8 at this concentration and time period results in posteriorization of the cells. The posteriorized NPCs produced at the end of this stage express more Hox genes, such as HoxA4, and have reduced expression of at least one of the brain markers such as Gbx2, Otx2 and FoxG1 compared to un-patterned cells (FIG. 6). Posteriorized NPCs are equally tripotent with the same differentiation profile as un-patterned NPCs. The ability to form neurospheres and the proliferation rate of posteriorized NPCs are marginally higher than un-patterned NPCs.
2) On day 3, use Accutase to passage cells to new PLL/laminin pre-coated standard culture plates in DMEM: F12 media supplemented with B27, N2, 0.1 μM EC23, and Wnt3a (100 μg/ml). Incubate for an additional 3 days.

In this step we induce caudalization of cells using retinoic acid (RA) or the synthetic retinoid analogue, EC23. Using EC23 is preferred as it is more photostable at incubation temperatures.

FGF and RA signaling are not sufficient (alone or together) to induce caudal characteristics in neural cells grown in vitro and Wnt signaling (Wnt3a) is further required to specify neural cells to a caudal identity⁴².
3) On day 6, use Accutase to passage cells to new PLL/laminin pre-coated standard culture plates in DMEM: F12 media supplemented with B27, N2 and 0.1 μM EC23. Incubate for an additional 2 days.

No Wnt3a is required at this stage.

Treatment with RA and Wnt for 3 days results in caudalization of cells. These caudalized NPCs express Hox genes such as HoxA4. EC23 is continued for an additional 2 days after passaging to stabilize the caudal identity of the NPCs. This additional RA pathway activation results in a significant reduction (to nearly no expression) of Gbx2, Otx2 and FoxG1 levels compared to posteriorized cells (FIG. 7). Caudalized NPCs are also tripotent with the same differentiation profile as primed NPCs. However, the ability of caudalized NPCs to form neurospheres and their proliferation rate are significantly reduced compared un-patterned NPCs (FIG. 7).
4) Passage Caudalized-NPCs for 2-3 passages in DMEM: F12 supplemented with B27, N2, FGF2 (10 ng/ml), EGF (10 ng/ml) and 740Y-P (1 μM) until the identity of cells get stabilized, at this stage spinal NPCs are formed. Spinal NPCs can be passed and maintained in this media for up 3-5 more passages for optimum results, but passing up to P10 (and beyond) can be acceptable depending on the culture conditions (FIG. 8).
during maintenance period, the proliferation rate of cells is reduced. To generate sufficient numbers of cells, prolonged culture for several passages is required. The concentration of FGF2 cannot be increased at this stage. To overcome this problem, 740Y-P is added which is as effective as FGF2 at promoting neuronal cell survival and proliferation via PI 3-kinase-Akt pathway⁴³. The effect of 740Y-P is dose dependent.
spNPCs between ˜P3-P10 can be used. Later passage cells may develop NPCs with mixed identity and cells that generate more GABA-ergic interneurons

After each passage, add 10 μM Rock inhibitor (Y-27632) on day 1 of culture.

TABLE 3
Materials

6-well Ultra-low Adherence Plates
740Y-P
Accutase
Dorsomorphin
Epidermal growth factor -like domain-containing protein 7 (EGFL-7)
Fibroblast Growth Factor-2 (FGF2)
Fibroblast Growth Factor-8b (FGF-8b)
Heparin
hPSC culture (hESCs or hiPSCs) (table1)
mTeSR Plus*
Laminin-511
Matrigel
Geltrex*
Neural Expansion media (NEM) (Table 2)
Neural Induction media (NIM) (Table 2)
Poly-L-lysine (PLL) 70-150 KDa
Recombinant Murine Wnt-3a
ReLeSR
Retinoic acid agonist EC23
ROCK inhibitor (Y-27632)
SB431542
Sonic hedgehog (Shh) agonist Purmorphamine

| Sequences | |
|---|---|
| Human Ndfip1 Sequence: | |
| 10 20 30 40 50 | |
| MALALAALAA VEPACGSRYQ QLQNEEESGE PEQAAGDAPP PYSSISAESA | |
| 60 70 80 90 100 | |
| AYFDYKDESG FPKPPSYNVA TTLPSYDEAE RTKAEATIPL VPGRDEDFVG | |
| 110 120 130 140 150 | |
| RDDFDDADQL RIGNDGIFML TFFMAFLFNW IGFFLSFCLT TSAAGRYGAI | |
| 160 170 180 190 200 | |
| SGFGLSLIKW ILIVRFSTYF PGYFDGQYWL WWVFLVLGFL LFLRGFINYA | |
| 210 220 | |
| KVRKMPETFS NLPRTRVLFI Y (prior art sequence; SEQ ID | |
| NO: 24) | |
| Codon optimized Ndfip1 peptide encoding sequence | |
| (SEQ ID NO: 2) | |
| ATGGCCCTGGCCCTGGCCGCCCTGGCCGCCGTGGAGCCCGCCTGCGGCAG | 50 |
| CCGCTACCAGCAGCTGCAGAACGAGGAGGAGAGCGGCGAGCCCGAGCAGG | 100 |
| CCGCCGGCGACGCCCCCCCCCCCTACAGCAGCATCAGCGCCGAGAGCGCC | 150 |
| GCCTACTTCGACTACAAGGACGAGAGCGGCTTCCCCAAGCCCCCCAGCTA | 200 |
| CAACGTGGCCACCACCCTGCCCAGCTACGACGAGGCCGAGCGCACCAAGG | 250 |
| CCGAGGCCACCATCCCCCTGGTGCCCGGCCGCGACGAGGACTTCGTGGGC | 300 |
| CGCGACGACTTCGACGACGCCGACCAGCTGCGCATCGGCAACGACGGCAT | 350 |
| CTTCATGCTGACCTTCTTCATGGCCTTCCTGTTCAACTGGATCGGCTTCT | 400 |
| TCCTGAGCTTCTGCCTGACCACCAGCGCCGCCGGCCGCTACGGCGCCATC | 450 |
| AGCGGCTTCGGCCTGAGCCTGATCAAGTGGATCCTGATCGTGCGCTTCAG | 500 |
| CACCTACTTCCCCGGCTACTTCGACGGCCAGTACTGGCTGTGGTGGGTGT | 550 |
| TCCTGGTGCTGGGCTTCCTGCTGTTCCTGCGCGGCTTCATCAACTACGCC | 600 |
| AAGGTGCGCAAGATGCCCGAGACCTTCAGCAACCTGCCCCGCACCCGCGT | 650 |
| GCTGTTCATCTAC | |

### REFERENCES

1. Park, K. K. et al. Promoting axon regeneration in the adult CNS by modulation of the PTEN/mTOR pathway. Science 322, 963-966 (2008).
2. Liu, K. et al. PTEN deletion enhances the regenerative ability of adult corticospinal neurons. Nat. Neurosci. 13, 1075-1081 (2010).
3. Sun, F. et al. Sustained axon regeneration induced by co-deletion of PTEN and SOCS3. Nature 480, 372-5 (2011).
4. Delgado-Esteban, M., Martin-Zanca, D., Andres-Martin, L., Almeida, A. & Bolaños, J. P. Inhibition of PTEN by peroxynitrite activates the phosphoinositide-3-kinase/Akt neuroprotective signaling pathway. J. Neurochem. 102, 194-205 (2007).
5. Domanskyi, A. et al. Pten ablation in adult dopaminergic neurons is neuroprotective in Parkinson's disease models. FASEB J. 25, 2898-2910 (2011).
6. Gregorian, C. et al. Pten deletion in adult neural stem/progenitor cells enhances constitutive neurogenesis. J. Neurosci. 29, 1874-1886 (2009).
7. Kurimoto, T. et al. Long-distance axon regeneration in the mature optic nerve: contributions of oncomodulin, cAMP, and pten gene deletion. J. Neurosci. 30, 15654-15663 (2010).
8. Christie, K. J., Webber, C. A., Martinez, J. A., Singh, B. & Zochodne, D. W. PTEN inhibition to facilitate intrinsic regenerative outgrowth of adult peripheral axons. J. Neurosci. 30, 9306-9315 (2010).
9. Nakashima, S. et al. Small-molecule protein tyrosine phosphatase inhibition as a neuroprotective treatment after spinal cord injury in adult rats. J. Neurosci. 28, 7293-7303 (2008).
10. Walker, C. L. et al. Systemic bisperoxovanadium activates Akt/mTOR, reduces autophagy, and enhances recovery following cervical spinal cord injury. PLOS ONE 7, e30012 (2012).
11. Takeuchi, K. et al. Dysregulation of synaptic plasticity precedes appearance of morphological defects in a Pten conditional knockout mouse model of autism. Proc. Natl. Acad. Sci. U.S.A. 110, 4738-4743 (2013).
12. Howitt, J. et al. Ndfip1 regulates nuclear Pten import in vivo to promote neuronal survival following cerebral ischemia. J. Cell Biol. 196, 29-36 (2012).
13. Fraser, M. M., Bayazitov, I. T., Zakharenko, S. S. & Baker, S. J. Phosphatase and tensin homolog, deleted on chromosome 10 deficiency in brain causes defects in synaptic structure, transmission and plasticity, and myelination abnormalities. Neuroscience 151, 476-488 (2008).
14. Sperow, M. et al. Phosphatase and tensin homologue (PTEN) regulates synaptic plasticity independently of its effect on neuronal morphology and migration. The Journal of Physiology 590, 777-792 (2012).
15. Backman, S. A. et al. Deletion of Pten in mouse brain causes seizures, ataxia and defects in soma size resembling Lhermitte-Duclos disease. Nat. Genet. 29, 396-403 (2001).
16. Jurado, S. et al. PTEN is recruited to the postsynaptic terminal for NMDA receptor-dependent long-term depression. EMBO J. 29, 2827-2840 (2010).
17. Diaz-Ruiz, O. et al. Selective deletion of PTEN in dopamine neurons leads to trophic effects and adaptation of striatal medium spiny projecting neurons. PLOS ONE 4, e7027 (2009).
18. Perandones, C. et al. Correlation between synaptogenesis and the PTEN phosphatase expression in dendrites during postnatal brain development. Brain Res. Mol. Brain Res. 128, 8-19 (2004).
19. Lackovic, J. et al. Differential regulation of Nedd4 ubiquitin ligases and their adaptor protein Ndfip1 in a rat model of ischemic stroke. Exp. Neurol. 235, 326-335 (2012).
20. Sang, Q. et al. Nedd4-WW domain-binding protein 5 (Ndfip1) is associated with neuronal survival after acute cortical brain injury. J. Neurosci. 26, 7234-7244 (2006).
21. Lachyankar, M. B. et al. A role for nuclear PTEN in neuronal differentiation. J. Neurosci. 20, 1404-1413 (2000).
22 Sorkin, A. & Goh, L. K. Endocytosis and intracellular trafficking of ErbBs. Exp. Cell Res. 315, 683-696 (2009).
23 Trotman, L. C. et al. Ubiquitination regulates PTEN nuclear import and tumor suppression. Cell 128, 141-156 (2007).
24. Fouladkou, F. et al. The ubiquitin ligase Nedd4-1 is dispensable for the regulation of PTEN stability and localization. Proc. Natl. Acad. Sci. U.S.A. 105, 8585-8590 (2008).
25. Wang, X. et al. NEDD4-1 is a proto-oncogenic ubiquitin ligase for PTEN. Cell 128, 129-139 (2007).
26 Mund, T. & Pelham, H. R. B. Regulation of PTEN/Akt and MAP kinase signaling pathways by the ubiquitin ligase activators Ndfip1 and Ndfip2. Proc. Natl. Acad. Sci. U.S.A. 107, 11429-11434 (2010).
27 Hammond, V. E. et al. Ndfip1 Is Required for the Development of Pyramidal Neuron Dendrites and Spines in the Neocortex. Cereb. Cortex bht191 (2013). doi: 10.1093/cercor/bht191
28. Schumacher, P. A., Eubanks, J. H. & Fehlings, M. G. Increased calpain I-mediated proteolysis, and preferential loss of dephosphorylated NF200, following traumatic spinal cord injury. Neuroscience 91, 733-744 (1999).
29. Hares, K. Neurofilament dot blot assays: Novel means of assessing axon viability in culture. Journal of Neuroscience Methods 198, 195-203 (2011).
30. Fotia, A. B. et al. Regulation of neuronal voltage-gated sodium channels by the ubiquitin-protein ligases Nedd4 and Nedd4-2. J. Biol. Chem. 279, 28930-28935 (2004).
31. Rougier, J.-S. et al. Molecular determinants of voltage-gated sodium channel regulation by the Nedd4/Nedd4-like proteins. Am. J. Physiol., Cell Physiol. 288, C692-701 (2005).
32. Agrawal, S. K. & Fehlings, M. G. Mechanisms of secondary injury to spinal cord axons in vitro: role of Na+, Na(+)-K(+)-ATPase, the Na(+)-H+ exchanger, and the Na(+)-Ca2+ exchanger. J. Neurosci. 16, 545-552 (1996).
33. Schwartz, G. & Fehlings, M. G. Secondary injury mechanisms of spinal cord trauma: a novel therapeutic approach for the management of secondary pathophysiology with the sodium channel blocker riluzole. Prog. Brain Res. 137, 177-190 (2002).

34. Khazaei, M. R & Fehlings, M. G. Down-regulating PTEN by Ndfip1 Treatment Promotes the Differentiation of Neural Progenitor Cells to Oligodendrocyte (poster presentation 2015). {Krembil Research Day, at Krembil Research Institute, Toronto}
35. Ohtake, Yosuke et al. “PTEN inhibition and axon regeneration and neural repair.” Neural regeneration research vol. 10,9 (2015): 1363-8.
36. Gilbert, Luke A et al. “CRISPR-mediated modular RNA-guided regulation of transcription in eukaryotes.” Cell vol. 154,2 (2013): 442-51.
37. Qi, Lei S et al. “Repurposing CRISPR as an RNA-guided platform for sequence-specific control of gene expression.” Cell vol. 152,5 (2013): 1173-83.
38. Lippmann ES, Williams CE, Ruhl DA, Estevez-Silva MC, Chapman ER, Coon JJ, Ashton RS: Deterministic HOX patterning in human pluripotent stem cell-derived neuroectoderm. Stem Cell Rep 2015, 4:632-644.
39. Delfino-Machín M, Lunn JS, Breitkreuz DN, Akai J, Storey KG: Specification and maintenance of the spinal cord stem zone. Dev Camb Engl 2005, 132:4273-4283.
40. Olivera-Martinez I, Harada H, Halley PA, Storey KG: Loss of FGF-dependent mesoderm identity and rise of endogenous retinoid signalling determine cessation of body axis elongation. PLOS Biol 2012, 10: e1001415.

1-38. (canceled)

39. A Ndfip1 fusion polypeptide comprising a neuron transport moiety and a Ndfip1 peptide.

40. The Ndfip1 fusion polypeptide of claim 39, wherein the neuron transport moiety is or comprises: (i) a neuron surface receptor ligand, (ii) an antibody, optionally a single domain antibody, or (iii) a Rabies Virus glycoprotein (RVG)-neuron permeabilization peptide, a translocation domain of diphtheria toxin (DTT), nontoxic C fragment of tetanus toxin (TTC), non-toxic pentameric b chain of the “Cholera toxin” (CTb), Neurotensin (NT) or Tet1 or an analog thereof that maintains the ability to facilitate transport into a neuron.

41. The Ndfip1 fusion polypeptide of claim 39, wherein the neuron transport moiety has the sequence of SEQ ID NO: 1, 2, 3 or 4 or at least 90% sequence identity to any of SEQ ID NO: 1, 2, 3, or 4.

42. A nucleic acid molecule encoding the Ndfip1 fusion polypeptide of claim 39, or a construct or expression cassette comprising said nucleic acid molecule.

43. The construct or expression cassette of claim 42, further comprising an inducible promoter.

44. The construct or expression cassette of claim 42, further comprising an export signal polynucleotide, optionally encoding any one of SEQ ID Nos: 6 to 23, preferably SEQ ID NO: 7 or 11.

45. The construct or expression cassette of claim 43, wherein the inducible promoter is a Tet-On inducible promoter, optionally TRE3G.

46. A cell expressing the Ndfip1 fusion polypeptide of claim 39, optionally secreting or inducibly secreting Ndfip1 fusion polypeptide.

47. The cell of claim 46, wherein the cell is:
(i) a neural lineage cell, optionally a neural progenitor cell (NPC), an oligodendrogenic NPC (oNPC), or a spinal identity NPC (spNPC);
(ii) a fibroblast or induced pluripotent stem cell (iPSC); or
(iii) a neural stem/progenitor cell, motor-neuron progenitor cell, differentiated neuron, neural stem cell, ventral neural progenitor cell, motor neuron progenitor (MNP), Motor Neral Progenitor Cell (pMN), Neuroepithelial precursor cell, or a central nervous system (CNS) neuronal cell type.

48. A therapeutic for use in treating a neurodegenerative disease and/or an optic nerve, brain, and/or spinal cord injury comprising the Ndfip1 fusion polypeptide of claim 39.

49. A therapeutic for use in treating a neurodegenerative disease and/or an optic nerve, brain, and/or spinal cord injury comprising the cell of claim 46.

50. A method of treating a neurodegenerative disease and/or an optic nerve, brain, and/or spinal cord injury in a subject in need thereof, the method comprising administering the Ndfip1 fusion polypeptide of claim 39 to the subject.

51. A method of treating a neurodegenerative disease and/or an optic nerve, brain, and/or spinal cord injury in a subject in need thereof, the method comprising administering the cell of claim 46 to the subject followed by an inducing agent, wherein the cell comprises an expression cassette or construct with an inducible promoter.

52. The method of claim 50, wherein the Ndfip1 fusion polypeptide is administered to or proximal to neurons damaged by the neurodegenerative disease and/or the optic nerve, brain, and/or spinal cord injury.

53. The method of claim 50, wherein the subject in need thereof has a neurodegenerative disease, an optic nerve, brain and/or spinal cord injury.

54. The method of claim 53, wherein the Ndfip1 fusion polypeptide is administered to the subject not earlier than two weeks following the optic nerve, brain and/or spinal cord injury.

55. A method of making a cell expressing a Ndfip1 fusion polypeptide comprising a neuron transport moiety and a Ndfip1 peptide, the method comprising the following steps:
a. inserting the nucleic acid, construct or expression cassette of claim 42 into a vector to make a vector construct; and
b. transfecting a cell with the vector construct.

56. A composition comprising the Ndfip1 fusion polypeptide of claim 39 and optionally a pharmaceutically acceptable carrier.

57. Use of the Ndfip1 fusion polypeptide of claim 39 in the manufacture of a medicament for treating a neurodegenerative disease and/or an optic nerve, brain, and/or spinal cord injury.

58. Use of the Ndfip1 fusion polypeptide of claim 39 to treat a neurodegenerative disease and/or an optic nerve, brain, and/or spinal cord injury.


SEQUENCE LISTING (29 sequences)

SEQ ID NO: 1 - protein, 30 aa, Rabies virus
YTIWMPENPRPGTPCDIFTNSRGKFRASNG

SEQ ID NO: 2 - DNA, 663 bp, Artificial Sequence
  Synthetic Construct
atggccctggccctggccgccctggccgccgtggagcccgcctgcggcagccgctaccagcagctgcagaacgaggaggagagcggcgagcccgagcaggccgccggcgacgccccccccccctacagcagcatcagcgccgagagcgccgcctacttcgactacaaggacgagagcggcttccccaagccccccagctacaacgtggccaccaccctgcccagctacgacgaggccgagcgcaccaaggccgaggccaccatccccctggtgcccggccgcgacgaggacttcgtgggccgcgacgacttcgacgacgccgaccagctgcgcatcggcaacgacggcatcttcatgctgaccttcttcatggccttcctgttcaactggatcggcttcttcctgagcttctgcctgaccaccagcgccgccggccgctacggcgccatcagcggcttcggcctgagcctgatcaagtggatcctgatcgtgcgcttcagcacctacttccccggctacttcgacggccagtactggctgtggtgggtgttcctggtgctgggcttcctgctgttcctgcgcggcttcatcaactacgccaaggtgcgcaagatgcccgagaccttcagcaacctgccccgcacccgcgtgctgttcatctac

SEQ ID NO: 3 - protein, 12 aa, Homo Sapiens
LYENKPRRPYIL

SEQ ID NO: 4 - protein, 13 aa, Artificial Sequence
  Synthetic Construct
HLNILSTLWKYRC

SEQ ID NO: 5 - protein, 15 aa, Artificial Sequence
  Synthetic Construct
GGGGSGGGGSGGGGS

SEQ ID NO: 6 - protein, 25 aa, Homo Sapiens
MGVLLTQRTLLSLVLALLFPSMASM

SEQ ID NO: 7 - protein, 16 aa, Vesicular stomatitis virus
MKCLLYLAFLFIGVNC

SEQ ID NO: 8 - protein, 21 aa, Mus musculus
METDTLLLWVLLLWVPGSTGD

SEQ ID NO: 9 - protein, 19 aa, Homo Sapiens
MGWSCIILFLVATATGVHS

SEQ ID NO: 10 - protein, 17 aa, Homo Sapiens
MRAWIFFLLCLAGRALA

SEQ ID NO: 11 - protein, 19 aa, Homo Sapiens
MEFGLSWVFLVALFRGVQC

SEQ ID NO: 12 - protein, 21 aa, Artificial Sequence
  Synthetic Construct
MWWRLWWLLLLLLLLWPMVWA

SEQ ID NO: 13 - protein, 22 aa, Homo Sapiens
MDMRVPAQLLGLLLLWLRGARC

SEQ ID NO: 14 - protein, 16 aa, Homo Sapiens
MPLLLLLPLLWAGALA

SEQ ID NO: 15 - protein, 23 aa, Homo Sapiens
MDAMKRGLCCVLLLCGAVFVSPS

SEQ ID NO: 16 - protein, 18 aa, Homo Sapiens
MAFLWLLSCWALLGTTFG

SEQ ID NO: 17 - protein, 15 aa, Homo Sapiens
MNLLLILTFVAAAVA

SEQ ID NO: 18 - protein, 20 aa, Homo Sapiens
MYRMQLLSCIALSLALVTNS

SEQ ID NO: 19 - protein, 17 aa, Gaussia Luciferase
MGVKVLFALICIAVAEA

SEQ ID NO: 20 - protein, 16 aa, Homo Sapiens
MKWVTFISLLFSSAYS

SEQ ID NO: 21 - protein, 16 aa, Influenza virus
MKTIIALSYIFCLVLG

SEQ ID NO: 22 - protein, 24 aa, Homo Sapiens
MALWMRLLPLLALLALWGPDPAAA

SEQ ID NO: 23 - protein, 16 aa, Bombyx mori
MKPIFLVLLVVTSAYA

SEQ ID NO: 24 - protein, 221 aa, Homo Sapiens
MALALAALAAVEPACGSRYQQLQNEEESGEPEQAAGDAPPPYSSISAESAAYFDYKDESGFPKPPSYNVATTLPSYDEAERTKAEATIPLVPGRDEDFVGRDDFDDADQLRIGNDGIFMLTFFMAFLFNWIGFFLSFCLTTSAAGRYGAISGFGLSLIKWILIVRFSTYFPGYFDGQYWLWWVFLVLGFLLFLRGFINYAKVRKMPETFSNLPRTRVLFIY

SEQ ID NO: 25 - protein, 24 aa, Artificial Sequence
  Synthetic Construct
GAPAPAKQEAPAPAKAEAPAPAKA

SEQ ID NO: 26 - protein, 15 aa, Artificial Sequence
  Synthetic Construct
  MISC_FEATURE (6)..(10): Can be either present or absent
  MISC_FEATURE (11)..(15): Can be either present or absent
EAAAKEAAAKEAAAK

SEQ ID NO: 27 - protein, 16 aa, Artificial Sequence
  Synthetic Construct
AEAAAKALEAEAAAKA

SEQ ID NO: 28 - protein, 18 aa, Artificial Sequence
  Synthetic Construct
KESGSVSSEQLAQFRSLD

SEQ ID NO: 29 - protein, 14 aa, Artificial Sequence
  Synthetic Construct
EGKSSGSGSESKST